(19) United States
(12) Patent Application Publication
(10) Pub. No.: US 2025/0145554 A1
(43) Pub. Date: May 8, 2025

(54) PROCESS FOR PROVIDING SYNTHESIS GAS AND FOR PRODUCING METHANOL

(71) Applicant: L'Air Liquide, Societe Anonyme pour l'Etude et l’Exploitation des Procedes Georges Claude, Paris (FR)

(72) Inventors: Tobias OELMANN, Frankfurt am Main (DE); Veronika GRONEMANN, Frankfurt am Main (DE)

(21) Appl. No.: 18/940,392

(22) Filed: Nov. 7, 2024

(30) Foreign Application Priority Data
Nov. 7, 2023 (EP) EP 23208276.8

(51) Int. Cl. C07C 29/151; C01B 3/36; C01B 3/38

(52) U.S. Cl. CPC C07C 29/1518; C01B 3/36; C01B 3/382; C01B 2203/0233; C01B 2203/025; C01B 2203/061; C01B 2203/1241; C01B 2203/1258; C01B 2203/142

(57) ABSTRACT

The present invention proposes a process for producing synthesis gas, in particular synthesis gas for methanol synthesis. The process includes the steps of providing a sulfur-containing hydrocarbon stream; providing an electrolytically produced hydrogen stream; supplying a portion of the electrolytically produced hydrogen stream to at least a portion of the sulfur-containing hydrocarbon stream to obtain a hydrogen-enriched sulfur-containing hydrocarbon stream; desulfurizing the stream obtained according to step (c) in a hydrodesulfurization unit (HDS unit) to obtain a sulfur-free hydrocarbon stream; supplying a portion of the electrolytically produced hydrogen stream to at least a portion of the stream obtained according to step (d) to obtain a hydrogen-enriched sulfur-free hydrocarbon stream and converting at least a portion of the stream obtained according to step (e) into a synthesis gas stream in the presence of oxygen as oxidant in a reforming step.

### CROSS-REFERENCE TO RELATED APPLICATIONS

This application claims the benefit of priority under 35 U.S.C. § 119 (a) and (b) to EP patent application No. EP 23208276.8, filed Nov. 7, 2023, the entire contents of which are incorporated herein by reference.

### BACKGROUND

### Field of the Invention

The invention relates to a process for producing synthesis gas. The invention further relates to a process for producing methanol comprising the abovementioned process for producing synthesis gas.

### Related Art

On a large industrial scale methanol is produced from synthesis gas. Synthesis gas is a mixture of predominantly hydrogen (H₂), carbon monoxide (CO) and carbon dioxide (CO₂). The term “carbon oxides” is often used to encompass carbon monoxide and carbon dioxide. Inter alia the following two equilibrium reactions (1) and (2) occur simultaneously over a solid methanol synthesis catalyst.

$$C {O}_{2} + 3 {H}_{2} ⇌ {CH}_{3} O H + {H}_{2} O \qquad ( 1 )$$

$$CO + 2 {H}_{2} ⇌ {CH}_{3} OH \qquad ( 2 )$$

The composition of the synthesis gas is characterized by the so-called stoichiometry number SN defined as

$$S N = \frac{n ( {H}_{2} ) - n ( {CO}_{2} )}{n ( CO ) + n ( {CO}_{2} )} , with n in [ mol ] .$$

A synthesis gas composition stoichiometrically balanced for methanol synthesis is characterized by a stoichiometry number SN of 2.0. Values of less than 2.0 indicate a hydrogen deficit while values of greater than 2.0 indicate a hydrogen excess. A synthesis gas having a stoichiometry number of less than 2.0 is also referred to as a substoichiometric synthesis gas.

Synthesis gases having a hydrogen deficit are generally obtained in processes comprising a partial oxidation step. These include partial oxidation (POX) as such and autothermal reforming (ATR).

The main elements of an ATR reactor are a burner, a combustion chamber and a catalyst bed in a refractory-lined pressure jacket. In an ATR reactor the partial oxidation of a hydrocarbon-containing input stream by substoichiometric amounts of oxygen is followed by a steam reforming of the partially oxidized hydrocarbon-containing input stream over a fixed bed of steam reforming catalyst.

For methane as the hydrocarbon a partial oxidation especially proceeds according to the following reaction equation (3).

$$C {H}_{4} + 1 \frac{1}{2} {O}_{2} ⇌ CO + 2 {H}_{2} O \qquad ( 3 )$$

The exothermic partial oxidation thus provides steam and the required heat energy for the endothermic steam reforming reaction which proceeds according to reaction equation (4).

$$C {H}_{4} + {H}_{2} O ⇌ 3 {H}_{2} + CO \qquad ( 4 )$$

Due to the high temperature the steam reforming (4) also occurs to a certain extent in the combustion chamber of the reactor without involvement of the catalyst.

The steam reforming reaction is accompanied by the water gas shift reaction according to reaction equation (5) which is likewise exothermic.

$$CO + {H}_{2} O ⇌ {CO}_{2} + {H}_{2} \qquad ( 5 )$$

The gas at the outlet of the ATR reactor is normally in or near to thermodynamic equilibrium in respect of the steam reforming and the water gas shift reaction.

The employed hydrocarbon often has steam added to it in an ATR. The precise composition of the synthesis gas at the outlet of the reactor depends on the hydrocarbon-to-steam ratio of the input stream, the process temperature and the process pressure. The temperature of the gas mixture at the outlet of the ATR reactor is typically in the range from 950° C. to 1050° C. The process pressure is typically 30 to 80 bar.

One alternative for producing synthesis gas is partial oxidation as such, also known as POX. The main elements of a POX reactor are a burner and a combustion chamber which are arranged in a refractory-lined pressure vessel. A POX reactor effects partial oxidation of a hydrocarbon-containing input stream due to substoichiometric amounts of oxygen. A certain (uncatalyzed) steam reforming also occurs and the water gas shift reaction accompanies the partial oxidation. The above reaction equations (3) to (5) are thus likewise applicable. The temperature of the gas mixture at the outlet of the reactor is typically in the range from 1250° C. to 1450° C. The process pressure is typically 30 to 100 bar.

As mentioned hereinabove the two above-described processes typically provide a markedly substoichiometric synthesis gas, i.e. a synthesis gas having a stoichiometry number of markedly below 2.0. Such a synthesis gas exhibits a hydrogen deficit in respect of the synthesis of methanol. If such a synthesis gas is directly employed for the production of methanol the hydrogen is thus virtually entirely consumed while a substantial portion of the carbon oxides is unconverted. This inter alia has the result that the content of byproducts (in particular higher alcohols and ketones) is higher than desired and that the yield of methanol is sub-optimal.

As a result, an industrial process generally supplies a stream of pure hydrogen to the stream of substoichiometric synthesis gas to increase the stoichiometry number SN of the synthesis gas to at least 1.9, preferably to 2.0 or more.

To this end conventional processes separate a portion of the reformed synthesis gas from the main stream and hydrogen from this substream is generally separated by pressure swing adsorption (PSA) or a membrane system. The tail gas generated in the pressure swing adsorption contains hydrocarbons and/or carbon monoxide and may therefore be utilized as fuel gas for the ATR or POX reactor.

If the hydrogen is instead provided by an electrolyzer it is possible to save a portion of the synthesis gas required for the methanol synthesis, thus improving the CO₂ footprint of the plant.

EP 3 658 494 B1 therefore proposes admixing hydrogen from an electrolyzer with a synthesis gas stream from a POX or ATR reactor. In addition, oxygen from an air separation unit is used as oxidant to operate the burner of the POX or ATR reactor.

The hydrogen provided by an electrolyzer typically contains residual oxygen in amounts of up to 1% by volume or more. This is associated with unavoidable diffusion processes occurring inside an electrolysis cell between the anode space and the cathode space. Inter alia a certain amount of the anodically produced oxygen always diffuses through the membrane or the diaphragm from the anode space into the cathode space, thus contaminating the cathodically produced hydrogen.

Oxygen represents a catalyst poison for methanol catalysts and therefore requires ideally quantitative removal from the cathodically produced hydrogen stream before said stream is admixed with the synthesis gas stream from the POX or ATR unit. This is effected in a so-called deoxygenation unit, de-ox unit for short. This comprises a catalytic stage for quantitative conversion of the contaminating oxygen with hydrogen from the hydrogen stream to afford water. The water thus catalytically produced may then be bound by adsorption, for example on a molecular sieve. This affords an oxygen- and water-free hydrogen stream.

The most important hydrocarbon-containing input material for the production of synthesis gas is natural gas. Natural gas generally contains sulfur compounds which are to be removed on account of their activity as catalyst poison in the ATR process or a downstream methanol synthesis. Desulfurization is typically carried out in a so-called hydrodesulfurization unit (HDS unit). Sulfur compounds such as mercaptans, sulfides, disulfides and thiophenes are hydrogenated over a catalyst at temperatures of 300° C. to 500° C. by supplying hydrogen, thus forming hydrogen sulfide as an easily removable sulfur compound. The hydrogen sulfide may subsequently be removed from the hydrocarbon mixture, for example by means of an amine scrubbing. It is customary to establish a hydrogen content of about 3% in the natural gas before the hydrogenation.

### SUMMARY OF THE INVENTION

It is an object of the present invention to propose a process which improves the integration of the aforementioned processes in such a way that especially carbon dioxide emissions are reduced in the production of synthesis gas and optionally in the subsequent synthesis of methanol.

It is a further object of the present invention to reduce the consumption of natural gas or other hydrocarbon-containing input materials in the production of synthesis gas and optionally a subsequent methanol synthesis.

The independent claims make a contribution to the at least partial achievement of at least one of the above objects. The dependent claims provide preferred embodiments which contribute to at least partial achievement of at least one of the objects. Preferred embodiments of constituents of one category according to the invention are, where relevant, likewise preferred for identically named or corresponding constituents of a respective other category according to the invention. The terms “having”, “comprising” or “containing” etc. do not preclude the possible presence of further elements, ingredients etc. The indefinite article “a” does not preclude the possible presence of a plurality.

The abovementioned objects are at least partially achieved by a process for producing synthesis gas, in particular synthesis gas for methanol synthesis, comprising the steps of:
(a) providing a sulfur-containing hydrocarbon stream;
(b) providing an electrolytically produced hydrogen stream;
(c) supplying a portion of the electrolytically produced hydrogen stream to at least a portion of the sulfur-containing hydrocarbon stream to obtain a hydrogen-enriched sulfur-containing hydrocarbon stream;
(d) desulfurizing the stream obtained according to step (c) in a hydrodesulfurization unit (HDS unit) to obtain a sulfur-free hydrocarbon stream;
(e) supplying a portion of the electrolytically produced hydrogen stream to at least a portion of the stream obtained according to step (d) to obtain a hydrogen-enriched sulfur-free hydrocarbon stream;
(f) converting at least a portion of the stream obtained according to step (e) into a synthesis gas stream in the presence of oxygen as oxidant in a reforming step.

According to the invention electrolytically produced hydrogen is utilized both for the desulfurization of the sulfur-containing hydrocarbon stream and for the enrichment of the produced synthesis gas with hydrogen. The latter is counterintuitively effected by supplying the hydrogen to the desulfurized hydrocarbon stream before the latter is supplied to the reforming step according to step (f), i.e. before instead of after production of synthesis gas.

The electrolytically produced hydrogen stream is utilized at least twice, namely for desulfurization of the sulfur-containing hydrocarbon stream and for enrichment of synthesis gas with hydrogen. If a correspondingly configured electrolyzer is used it is unnecessary to separate a synthesis gas substream from the process for the sole purpose of producing hydrogen in order to subsequently supply this hydrogen to the synthesis gas main stream for enrichment with hydrogen. This markedly reduces the proportion of hydrocarbon-containing process gas.

The at least twofold utilization of electrolytically produced hydrogen also allows the required electrolyzer to be utilized more flexibly. If less electrolytically produced hydrogen is available due to variation in the provision of renewable energy this smaller amount may be utilized for example entirely for the desulfurization and only partially for the enrichment of the synthesis gas with hydrogen. The hydrogen amount lacking for the hydrogen-enrichment of the synthesis gas may then be provided for example by a hydrogen recovery unit such as a PSA unit (pressure swing adsorption unit) which is typically part of a downstream methanol synthesis. There, hydrogen may be separated from the purge stream using a hydrogen recovery plant, for example a PSA unit or a membrane system.

The electrolytically produced hydrogen stream is counterintuitively supplied to the sulfur-free hydrocarbon stream according to step (e) before the conversion of the hydrocarbon stream into synthesis gas according to step (f). The electrolytically produced hydrogen stream is supplied to the sulfur-free hydrocarbon stream especially upstream of the respective reactor configured for the reforming step. This reactor is in particular a POX reactor or an ATR reactor. The resulting hydrogen-enriched sulfur-free hydrocarbon stream is then converted into synthesis gas in the presence of oxygen as oxidant according to step (f).

It is therefore possible according to the invention to use an oxygen-containing electrolytically produced hydrogen stream for the conversion according to step (f). If the electrolytically produced hydrogen stream were supplied to the synthesis gas stream only after the conversion according to step (f) as per the prior art this would require a quantitative removal of oxygen from the electrolytically produced hydrogen stream. As described above oxygen represents a catalyst poison for methanol synthesis catalysts and other catalyst types.

The reforming step according to step (f) is thus necessarily a reforming step in which oxygen is employed in a substoichiometric amount relative to the employed hydrocarbon. If this were not the case it would not be possible to produce a synthesis gas stream from the hydrogen-containing input gas stream. The employed oxygen is thus preferably completely consumed according to step (f) so that the produced synthesis gas stream is free from oxygen.

In one example a portion of the electrolytically produced hydrogen stream according to step (c) is supplied to the complete sulfur-containing hydrocarbon stream.

In one example a portion of the electrolytically produced hydrogen stream according to step (e) is supplied to the complete sulfur-free hydrocarbon stream produced according to step (d).

In a further example the electrolytically produced hydrogen stream, divided over two substreams, is entirely utilized for steps (c) and (e).

The sulfur-containing hydrocarbon stream is preferably a natural gas stream comprising methane as the primary component. The methane preferably accounts for a proportion of at least 50% by volume, preferably at least 75% by volume, more preferably at least 90% by volume, more preferably at least 95% by volume, more preferably at least 99% by volume.

The stream obtained according to step (c) preferably has a hydrogen content of 1% to 10% by volume, preferably of 1% to 5% by volume, more preferably of 2% to 4% by volume.

The electrolytically produced hydrogen stream may be produced by any electrolysis process familiar to those skilled in the art. The electrolysis process is preferably a water electrolysis. Examples include alkaline electrolysis, proton exchange membrane electrolysis (PEM electrolysis), anion exchange membrane electrolysis (AEM electrolysis), high-temperature electrolysis (HTE) and electrolysis using solid oxide electrolyzer cells (SOEC).

The synthesis gas stream comprises synthesis gas. A synthesis gas is a gas mixture which comprises at least a carbon oxide (carbon monoxide or carbon dioxide) and hydrogen. The synthesis gas preferably comprises carbon monoxide, carbon dioxide and hydrogen.

A reforming step is in principle to be understood as meaning the chemical reaction of hydrocarbons with oxygen and/or steam to afford a synthesis gas.

A preferred embodiment of the process according to the invention is characterized in that the electrolytically produced hydrogen stream contains oxygen as a concomitant, wherein
no oxygen is removed from the portion of the electrolytically produced hydrogen stream supplied to the sulfur-free hydrocarbon stream according to step (e) and
the oxygen is removed from the portion of the electrolytically produced hydrogen stream supplied to the sulfur-containing hydrocarbon stream according to step (c).

As mentioned above the hydrocarbon stream enriched with electrolysis hydrogen provided for the reforming with oxygen need not be free from oxygen. A portion of the electrolytically produced hydrogen stream may therefore be supplied to the sulfur-free hydrocarbon stream as a raw hydrogen stream in the sense of step (e). The removal of water from this hydrogen substream is likewise not required since especially in an ATR process according to the above reaction equations steam is formed as well as consumed.

A preferred embodiment of the process according to the invention is characterized in that the reforming step comprises an autothermal reforming (ATR) or a partial oxidation (POX) of the stream obtained according to step (e). It is preferable when the reforming step is an autothermal reforming.

A preferred embodiment of the process according to the invention is characterized in that
a portion of the stream obtained according to step (d) is converted into a synthesis gas substream in an endothermic steam reforming step and
a portion of the stream obtained according to step (d) is enriched with hydrogen according to step (e) to obtain a hydrogen-enriched sulfur-free hydrocarbon substream and
the aforementioned substreams are combined and converted into the synthesis gas stream in a reforming step in the presence of oxygen as oxidant according to step (f).

The endothermic steam reforming step is preferably an SMR (steam methane reforming) process. The reforming step in the presence of oxygen is preferably an ATR process.

In the aforementioned embodiment a portion of the sulfur-free hydrocarbon stream is subjected to an endothermic steam reforming step without enrichment with hydrogen. The resulting synthesis gas is combined with a hydrogen-enriched sulfur-free hydrocarbon stream. The resulting mixed stream which already contains synthesis gas from the endothermic steam reforming process is subjected to the reforming step according to step (f) in the presence of oxygen as oxidant.

In the present context the reforming step is especially an autothermal reforming step. This autothermal reforming step has an endothermic steam reforming step in which a portion of the sulfur-free hydrocarbon stream is reformed with steam arranged upstream of it. This procedure in principle affords a synthesis gas having a higher stoichiometry number than is obtained in a pure ATR process. As a result a smaller amount of hydrogen is required to adjust the stoichiometry number of the synthesis gas to a value suitable for methanol synthesis. The electrolyzer may accordingly be made smaller.

Alternatively, the excess electrolytically produced hydrogen may be utilized for under-firing of the endothermic steam reforming process.

A preferred embodiment the process is therefore characterized in that a portion of the electrolytically produced hydrogen stream provided according to step (b) is utilized as fuel in the endothermic steam reforming step.

In this context it is preferred that the electrolytically produced hydrogen stream contains oxygen as a concomitant and that no oxygen is removed from the portion of the electrolytically produced hydrogen stream utilized as fuel in the endothermic steam reforming step.

The removal of oxygen from an electrolysis hydrogen substream utilized for under-firing of a steam reformer is unnecessary since combustion requires the supply of combustion air or oxygen.

In this context a preferred embodiment of the process is characterized in that the portion of the electrolytically produced hydrogen stream utilized as fuel is combined with a portion of the sulfur-containing hydrocarbon stream provided according to step (a) to obtain a hydrogen- and hydrocarbon-containing mixed fuel stream which is utilized as fuel in the endothermic steam reforming step. It is alternatively also possible to utilize a portion of the desulfurized hydrocarbon stream for the mixed fuel stream. In this case a desulfurization of the flue gases formed by the endothermic steam reforming process (removal of sulfur oxides) is unnecessary.

A preferred embodiment of the process according to the invention is characterized in that an amount of electrolytically produced hydrogen supplied to the sulfur-free hydrocarbon stream according to step (e) is adjusted such that a synthesis gas stream obtained according to step (f) has a stoichiometry number SN of 1.9 to 2.5, preferably of 2.0 to 2.4, wherein

$$S N = \frac{n ( {H}_{2} ) - n ( {CO}_{2} )}{n ( CO ) + n ( {CO}_{2} )} , with n in [ mol ] .$$

This ensures that a synthesis gas stream which is directly, i.e. immediately, employable for a downstream methanol synthesis is obtained. The resulting synthesis gas stream can at least be used for a downstream methanol synthesis without any need for a further supply of hydrogen to the synthesis gas stream. The resulting synthesis gas stream is likewise employable immediately or at least without further supply of hydrogen for other syntheses which require synthesis gas compositions having a corresponding stoichiometry number.

A preferred embodiment of the process according to the invention is characterized in that the electrolytically produced hydrogen stream contains up to 5% by volume of oxygen or 0.01% to 5% by volume of oxygen or 0.1% to 3% by volume of oxygen or 0.1% to 1% by volume of oxygen as a concomitant.

A preferred embodiment of the process according to the invention is characterized in that the process comprises providing an electrolytically produced oxygen stream, wherein the electrolytically produced oxygen stream is used as oxidant in step (f).

The reforming step according to step (f) in principle proceeds in the presence of oxygen as oxidant. Air, oxygen-enriched air or pure oxygen may be employed here. The oxidant is in particular supplied to the burner of a POX or ATR unit and the hydrogen-enriched hydrocarbon stream is reacted therein with a substoichiometric amount of oxygen to afford synthesis gas.

An electrolyzer typically produces oxygen as a “byproduct”, wherein this byproduct is often not utilized. The use of the electrolytically produced oxygen as oxidant for the reforming step according to step (f) thus represents an improvement in the process integration of the process according to the invention.

A further embodiment of the process according to the invention is characterized in that the process comprises providing an oxygen stream produced by air separation, wherein the oxygen stream produced by air separation is used as oxidant in step (f).

Alternatively or in addition to the use of electrolysis oxygen the process may comprise providing an oxygen stream produced by air separation, wherein the oxygen stream produced by air separation is used as oxidant in step (f).

Alternatively or in addition the oxygen may also be provided to the process via a pipeline.

A preferred embodiment of the process according to the invention is characterized in that the conversion of at least a portion of the stream obtained according to step (e) in the presence of oxygen as oxidant in a reforming step to afford a synthesis gas stream according to step (f) is carried out with additional supply of steam.

A steam stream is optionally supplied to the sulfur-free hydrocarbon stream before the conversion according to step (f), in particular when the reforming step comprises an autothermal reforming (ATR).

Additional steam is preferably supplied when the reforming step comprises an autothermal reforming. This advantageously prevents carbon deposits on the catalyst utilized for the endothermic steam reforming in the ATR reactor. The term “additional steam” is to be understood as meaning steam which is not produced reaction-internally by the partial oxidation reaction or the reaction of hydrogen to afford water. Depending on whether the supplying of the steam stream is effected before or after the supplying of the electrolytically produced hydrogen stream to the hydrocarbon-containing input gas stream, addition of a steam stream accordingly results in a steam-containing and hydrogen-enriched hydrocarbon stream.

A preferred embodiment of the process according to the invention is characterized in that the electrolytically produced hydrogen stream is partially supplied to the synthesis gas stream produced according to step (f).

It is preferable to remove the oxygen from this portion of the electrolytically produced hydrogen stream when the thus-produced synthesis gas stream is subjected to a subsequent methanol synthesis. It is particularly preferable when the oxygen is removed from a substream of the electrolytically produced hydrogen stream and this oxygen-free hydrogen substream is subsequently divided over two further hydrogen substreams. The first of these substreams is supplied to at least a portion of the sulfur-containing hydrocarbon stream according to step (c). The second of these substreams is supplied to the synthesis gas stream produced according to step (f) according to the aforementioned embodiment. The synthesis gas stream enriched with oxygen-free hydrogen is thus preferably subjected to a subsequent synthesis, preferably subjected to a methanol synthesis.

The abovementioned objects are further at least partially achieved by a process for producing methanol comprising the process for producing synthesis gas according to any of the abovementioned embodiments, further comprising the step of reacting the synthesis gas stream over a solid methanol synthesis catalyst to afford raw methanol, wherein the raw methanol comprises at least methanol (CH₃OH) and water.

The produced synthesis gas stream is preferably converted into raw methanol over a solid methanol synthesis reactor directly, in particular without a further modification step, in particular without further supplying of a hydrogen stream. The synthesis gas of the synthesis gas stream used for the methanol synthesis preferably has a stoichiometry number SN of 1.9 to 2.5, preferably of 2.0 to 2.4.

The synthesis gas of the synthesis gas stream employed for the methanol synthesis and produced according to the invention may also be referred to as synthesis make-up gas. This synthesis gas is in most cases distinguishable in terms of its composition from the synthesis gas which is actually introduced into the respective methanol synthesis reactor. As is known to those skilled in the art the methanol synthesis is on the large industrial scale generally configured as a synthesis loop, i.e. synthesis gas unreacted in the methanol synthesis reactor is recycled to the reactor inlet after separation of condensed-out raw methanol. This recycled synthesis gas, also known as recycle gas or recirculated gas, is mixed with the synthesis make-up gas. The resulting mixed synthesis gas having the composition as at the reactor inlet may have a stoichiometry number diverging from the abovementioned interval for the synthesis make-up gas.

A preferred embodiment of the process according to the invention for producing methanol is characterized in that the raw methanol is separated into pure methanol and water in a thermal separation process, wherein the thermal separation process affords a carbon-containing tail gas stream, wherein the carbon-containing tail gas stream is utilized as fuel in a heating apparatus for preheating the stream obtained according to step (e) before said stream is converted into synthesis gas according to step (f).

The distillation of raw methanol to obtain pure methanol typically generates a stream of low-boiling byproducts-referred to here as a tail gas stream-which may be utilized as a fuel in a heating apparatus for preheating the stream obtained according to step (e). It is preferable when the stream obtained according to step (e) is preheated optionally together with steam before being subjected to the actual reforming step according to step (f). The heating apparatus is especially a fired heating apparatus.

The aforementioned utilization of the tail gas stream generated in the distillation of raw methanol further improves process integration.

A preferred embodiment of the process according to the invention for producing methanol is characterized in that the water separated in the thermal separation process is used as starting material for the electrolytically produced hydrogen stream.

It is preferable when the water separated in the thermal separation process is worked up and subsequently used as starting material for the electrolytically produced hydrogen. The water separated in the thermal separation process often contains sodium hydroxide (NaOH) which must be removed for the utilization of the water in a PEM electrolysis for example. If the electrolytically produced hydrogen stream is produced by an alkaline electrolysis the removal of sodium hydroxide is not absolutely necessary since the alkaline electrolysis employs highly concentrated aqueous potassium hydroxide solution (KOH_{aq}) or aqueous sodium hydroxide solution (NaOH_{aq}) as electrolysis medium.

The utilization of the water separated in the thermal separation process as starting material for the electrolytically produced hydrogen improves the process integration since fewer resources are required for the provision of water for the water electrolysis.

A preferred embodiment of the process according to the invention for producing methanol is characterized in that reacting the synthesis gas stream over the solid methanol synthesis catalyst to afford raw methanol generates a residual gas stream containing synthesis gas unconverted into raw methanol, wherein a portion of the residual gas stream is separated as a purge gas stream and wherein the purge gas stream is supplied to a hydrogen recovery apparatus to produce a non-electrolytically produced hydrogen stream and
the non-electrolytically produced hydrogen stream is at least partially additionally supplied to the sulfur-containing hydrocarbon stream to obtain the hydrogen-enriched sulfur-containing hydrocarbon stream and/or
the non-electrolytically produced hydrogen stream is at least partially additionally supplied to the stream obtained according to step (d) to obtain the hydrogen-enriched sulfur-free hydrocarbon stream.

As mentioned hereinabove, on an industrial scale the synthesis of methanol is typically carried out within a so-called synthesis loop. The reaction of the synthesis gas over the methanol synthesis catalyst is incomplete due to the establishment of a thermodynamic equilibrium and not only condensable raw methanol but also a non-condensable residual gas stream containing unconverted synthesis gas are obtained at the outlet of the respective reactor. A portion of this residual gas stream is recycled to the reactor inlet for renewed reaction to afford raw methanol. This recycled stream is referred to as the recycle gas stream or recirculated gas stream. A portion is separated as a purge gas stream to avoid accumulation of compounds inert under the conditions of methanol synthesis in the synthesis loop.

This purge gas stream containing hydrogen from the unconverted synthesis gas (residual gas stream) is advantageously supplied to a hydrogen recovery apparatus in order thus to produce a non-electrolytically produced hydrogen stream. The hydrogen recovery apparatus is preferably a pressure swing adsorption apparatus (PSA). It may alternatively also be a membrane unit.

The non-electrolytically produced hydrogen stream may be at least partially additionally supplied to the sulfur-containing hydrocarbon stream to obtain the hydrogen-enriched sulfur-containing hydrocarbon stream. Alternatively or in addition the non-electrolytically produced hydrogen stream may be at least partially additionally supplied to the stream obtained according to step (d) to obtain the hydrogen-enriched sulfur-free hydrocarbon stream.

The non-electrolytically produced hydrogen stream may thus augment the electrolytically produced hydrogen stream, either in the production of the hydrogen-enriched sulfur-containing hydrocarbon stream and/or in the production of the hydrogen-enriched sulfur-free hydrocarbon stream. In the latter case sufficient non-electrolytically produced hydrogen is supplied together with the electrolytically produced hydrogen stream to ensure that step (f) in turn preferably affords a synthesis gas having a stoichiometry number SN of 1.9 to 2.5, preferably having a stoichiometry number SN of 2.0 to 2.4.

Alternatively or in addition the non-electrolytically produced hydrogen stream may be supplied to the synthesis gas stream downstream of the respective reforming step with oxygen as oxidant (POX or ATR) and upstream of the methanol synthesis.

Since a methanol plant having a synthesis loop always generates a purge gas stream this may advantageously be utilized process-internally. This especially makes it possible to compensate for the variations in the hydrogen production of an electrolyzer, for example when this electrolyzer produces hydrogen based on renewable electricity which is not available on a constant basis over time.

### BRIEF DESCRIPTION OF THE FIGURES

The invention is more particularly elucidated hereinbelow by way of exemplary embodiments and a numerical example without in any way limiting the subject matter of the invention. In the figures, functionally and/or structurally identical or at least similar constituents are given identical reference numerals.

In the figures:

FIG. 1 shows a block flow diagram of the inventive process according to a first example and

FIG. 2 shows a block flow diagram of the inventive process according to a second example.

### DETAILED DESCRIPTION OF THE INVENTION

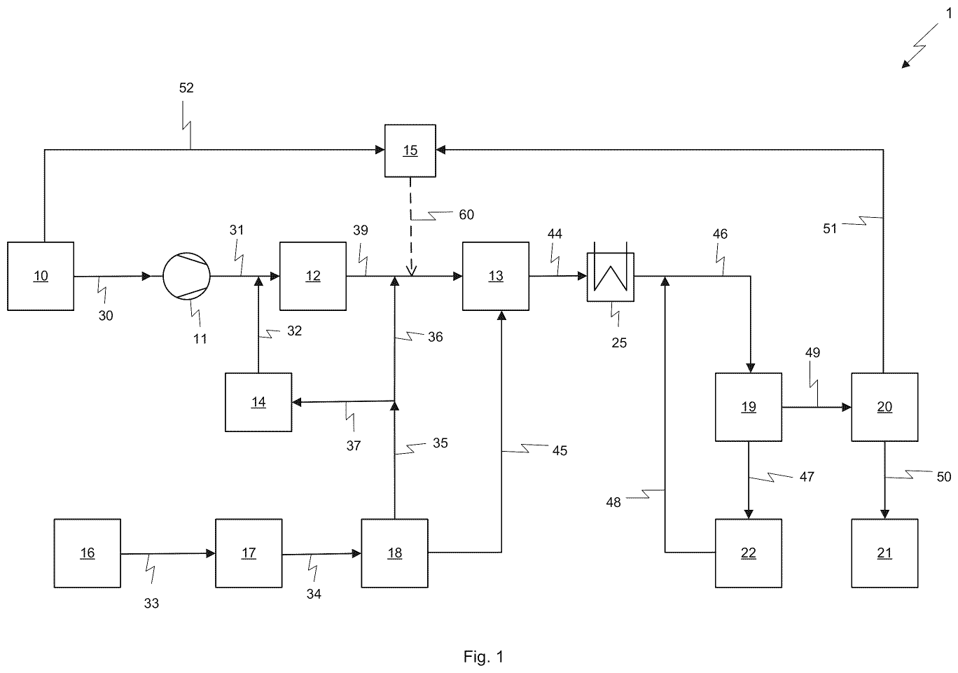

FIG. 1 shows a simplified block flow diagram of a process 1 for synthesis gas production and subsequent methanol production according to a first example of the invention.

From a natural gas source 10 which contains primarily methane as the hydrocarbon, a sulfur-containing hydrocarbon stream is initially supplied to a compressor 11 via a conduit 30 and subsequently sent on in compressed form via conduit 31.

From a water source 16 raw water is supplied to a water treatment unit 17 via a conduit 33. In the water treatment unit 17 the raw water is treated such that it is suitable for a subsequent water electrolysis. The treatment comprises for example a filtration, the removal of dissolved salts and a degassing. The treated water is supplied via conduit 34 to an electrolyzer 18 which may be a PEM electrolyzer for example. The electrolysis medium is in this case the water treated by the water treatment unit 17.

The electrolyzer 18 produces a hydrogen stream and an oxygen stream. The hydrogen stream is withdrawn from the electrolyzer 18 via a conduit 35 and subsequently divided over two substreams. The first substream is sent on via a conduit 37 and supplied to a deoxygenation unit 14. In the deoxygenation unit 14 the electrolytically produced hydrogen stream is freed of oxygen. This hydrogen stream freed of oxygen is sent on via a conduit 32 and mixed with the sulfur-containing hydrocarbon stream in conduit 31, thus resulting in a hydrogen-enriched hydrocarbon stream in conduit 31. This hydrogen-enriched hydrocarbon stream comprises a hydrogen content of about 3% by volume. The hydrogen-enriched sulfur-containing hydrocarbon stream is sent on into a hydrodesulfurization unit 12 (HDS unit 12) in which the hydrocarbon stream is freed of sulfur compounds.

The thus-desulfurized hydrocarbon stream is discharged from the HDS unit 12 via a conduit 39.

The second hydrogen substream produced in the electrolyzer 18 is sent on via a conduit 36 and not subjected to a further purification by a deoxygenation unit. This hydrogen substream is supplied to a conduit 39 via conduit 36 to obtain in this conduit 39 a hydrogen-enriched sulfur-free hydrocarbon stream. The supplied hydrogen amount is adjusted such that the subsequent reforming reaction affords a synthesis gas having a predetermined stoichiometry number SN.

The electrolyzer 18 also produces an oxygen stream which is discharged from the electrolyzer 18 via a conduit 45. This oxygen stream is supplied to an ATR reactor 13 (i.e. autothermal reformer 13). The hydrogen-enriched sulfur-free hydrocarbon stream from conduit 39 is also supplied to the ATR reactor 13. The ATR reactor 13 reforms the aforementioned streams into a synthesis gas stream which is discharged from the ATR reactor via conduit 44. The synthesis gas stream produced in the ATR reactor 13 is cooled in a thermal recovery unit 25. The thermal recovery unit 25 simultaneously produces steam which may optionally be utilized for reforming the hydrogen-enriched hydrocarbon stream in the ATR reactor 13 (not shown).

The cooled synthesis gas stream is discharged from the thermal recovery unit 25 via conduit 46. Said stream is subsequently supplied via the same conduit 46 to a methanol synthesis unit 19 which comprises at least a reactor having a solid methanol synthesis catalyst, a condenser and a gas/liquid separator for producing raw methanol (not shown). In the methanol synthesis unit 19 unconverted synthesis gas is recycled to the reactor inlet as recirculated gas (not shown). A proportion of this recirculated gas stream is withdrawn as a purge gas stream to prevent enriching of components inert under the conditions of methanol synthesis in the methanol synthesis unit 19. This purge gas stream is discharged from the methanol synthesis unit 19 via a conduit 47 and subsequently supplied to a hydrogen recovery unit, here a PSA unit 22. A hydrogen stream, which in this case is not electrolytically produced, and a tail gas stream are produced from the purge gas stream in the PSA unit 22. The hydrogen stream is discharged from the PSA unit via a conduit 48 and supplied to the cooled synthesis gas stream in conduit 46. The non-electrolytically produced hydrogen stream is thus used for adjusting the stoichiometry number, desired for the methanol synthesis, of the synthesis gas supplied to the methanol synthesis unit 19. The stoichiometry number desired for the methanol synthesis could equally be adjusted solely by supplying the electrolytically produced hydrogen stream from conduit 36 to the conduit 39. In this case the hydrogen produced in the PSA unit may be otherwise utilized.

The raw methanol produced in the methanol synthesis unit 19 comprises substantially methanol, water and undesired byproducts. The raw methanol is supplied to a distillation unit 20 via a conduit 49 for thermal separation into the desired components. The distillation unit 20 may comprise one or more distillation columns. The distillation unit 20 produces at least pure methanol in a prespecified purity and water. The produced water may be supplied to the water treatment unit 17 to be utilized as starting material in the electrolyzer 18. The distillation unit 20 also produces a carbon-containing tail gas stream of low-boiling substances. This tail gas stream is discharged from the distillation unit 20 via a conduit 51 and supplied to a fired heating means 15. The fired heating means 15 is used for preheating the hydrogen-enriched sulfur-free hydrocarbon stream before it is supplied to the ATR reactor 13. To this end the fired heating means 15 produces a heat flux 60 indicated by the dashed arrow. The fired heating means 15 is also supplied with a portion of the hydrocarbon stream deriving from the natural gas source 10. To reduce the carbon dioxide emissions of the fired heating means 15 this hydrogen also not required in the process from the electrolyzer 18 or the PSA unit 22 may be supplied as fuel.

The pure methanol produced in the distillation unit 20 is withdrawn from the distillation unit 20 via a conduit 50 and subjected to a further utilization as methanol product 21.

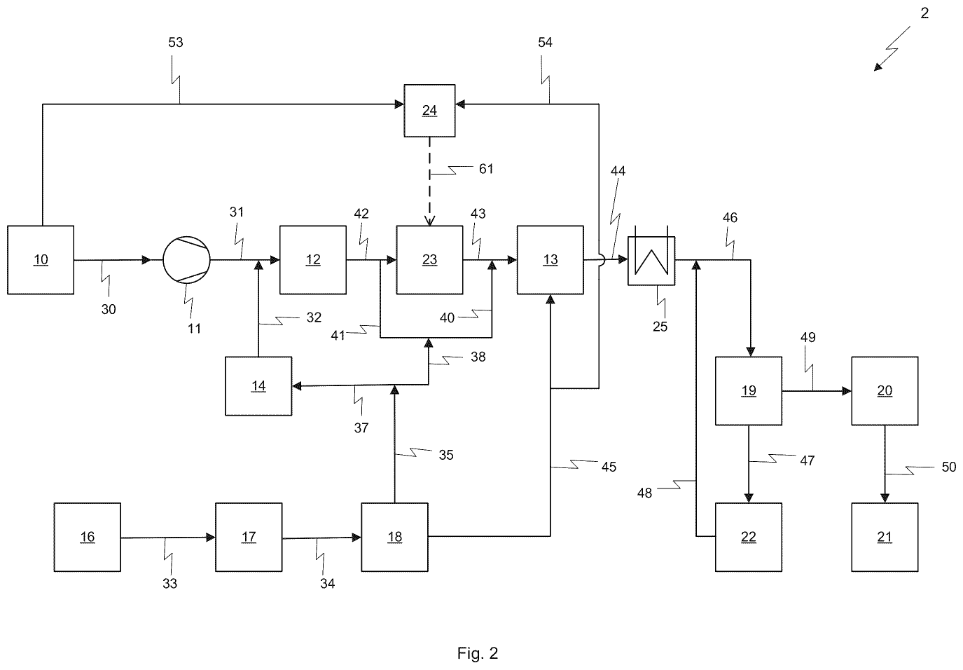

FIG. 2 shows a simplified block flow diagram of a process 2 for synthesis gas production and subsequent methanol production according to a second example of the invention. The following addresses substantially the differences of process 2 relative to process 1.

In process 2 according to FIG. 2 2 a sulfur-free hydrocarbon stream is withdrawn from the HDS unit 12 via a conduit 42. A substream of the desulfurized hydrocarbon stream is withdrawn from this conduit 42 via a conduit 41. The remaining hydrocarbon substream is supplied to an SMR unit 23 via conduit 42 and therein subjected to an endothermic steam reforming.

Just as in the example according to FIG. 1 the electrolyzer 18 produces a hydrogen stream which is divided over two substreams in a conduit 37 and a conduit 38. A portion of the sulfur-free hydrocarbon stream is withdrawn from conduit 42 and sent on via a conduit 41. The hydrogen substream in conduit 38 is supplied to this hydrocarbon substream in conduit 41 to obtain a hydrogen-enriched sulfur-free hydrocarbon substream. This is sent on via a conduit 40 and in conduit 43 combined with a synthesis gas substream discharged from the SMR unit 23 as a product of the endothermic steam reforming. This results in conduit 43 in a mixture of synthesis gas and hydrogen-enriched hydrocarbon. This mixture is subjected to a further a reforming step, in this case in the presence of oxygen, in the ATR reactor 13. Analogously to the example of FIG. 1 the oxygen for the reforming in the ATR reactor 13 is supplied by an oxygen stream provided by the electrolyzer and supplied to the ATR reactor via conduit 45.

The endothermicity of the steam reforming reaction means that the SMR unit 23 requires an SMR firing means 24 which is typically composed of two or more rows of burners which are arranged in rows for firing catalyst-filled reformer tubes in a reformer furnace known to those skilled in the art. The oxygen required for the burner is diverted from conduit 45 and supplied to the SMR firing means 24 via a conduit 54. Fuel required therefor is at the same time withdrawn from the natural gas source 10 and supplied to the SMR firing means via conduit 53. The thermal energy provided by the SMR firing means is shown as the heat flux 61 flowing towards the SMR unit in the form of a dashed arrow.

Reforming of the synthesis gas substream provided by the SMR unit 23 and the hydrogen-enriched sulfur-free hydrocarbon stream in the ATR reactor 13 affords a synthesis gas stream whose composition has a greater stoichiometry number than the synthesis gas stream obtainable by the process of FIG. 1. This synthesis gas stream accordingly requires less hydrogen to be supplied to it from the PSA unit 22 via conduit 48 and/or the hydrocarbon stream in conduit 41 requires less hydrogen to be supplied to it via conduit 38 from the outset.

The following numerical example is based on simulation data and serves to further elucidate the invention.

The following table shows simulation data of two comparative examples 1 and 2 and of an inventive example.

| | | Comparative | Comparative | Inventive |
|---|---|---|---|---|
| | Unit | example 1 | example 2 | example |
| Total natural gas consumption | kmol/h | 4360 | 4316 | 4086 |
| Natural gas to process | kmol/h | 4065 | 4065 | 3573 |
| Natural gas to fired heating means | kmol/h | 139 | 180 | 308 |
| Natural gas to steam generator | kmol/h | 156 | 70 | 205 |
| Product amount | t/h | 125 | 125 | 125 |
| Synthesis gas to PSA | kmol/h | 1980 | 1480 | 0 |
| Hydrogen produced by PSA | kmol/h | 1433 | 1243 | 318 |
| Electrolytically produced hydrogen | kmol/h | 0 | 126 | 808 |
| Carbon dioxide emissions of fired | kg/h | 44663 | 42066 | 28033 |
| heating means | | | | |
| Carbon dioxide emissions of steam | kg/h | 8033 | 8412 | 10571 |
| generator | | | | |
| Natural gas saving | % | n/a | 1.02 | 6.28 |

Each of the examples in principle produces 3000 t of methanol per day, wherein an autothermal reformer (ATR) is used to produce the synthesis gas.

In comparative example 1, a portion of the reformed gas (synthesis gas) is used to obtain hydrogen in a PSA unit in order thus to obtain hydrogen for adjusting the stoichiometry number of the produced synthesis gas and for the HDS unit. This hydrogen is accordingly produced exclusively by a PSA which may also obtain purge gas from methanol synthesis as starting material. Comparative example 1 thus does not produce hydrogen by electrolysis.

According to comparative example 2 the majority of the required hydrogen is further produced by a PSA unit which is supplied with reformed gas (synthesis gas). This is used for adjusting the stoichiometry number of the produced synthesis gas for the methanol synthesis. The hydrogen required for the HDS unit is provided by an electrolyzer.

In the inventive example the majority of the required hydrogen is provided by an electrolyzer. The PSA unit produces hydrogen exclusively from the purge gas of the methanol synthesis. No reformed gas (synthesis gas) is supplied to it. Accordingly the majority of the hydrogen required for adjusting the stoichiometry number of the synthesis gas and for the HDS unit is provided by the electrolyzer. This procedure achieves corresponding natural gas savings of more than 5% relative to the second comparative example and more than 6% relative to the first comparative example. It is surprisingly also shows advantages in terms of the carbon dioxide emissions of the employed fired heating apparatus. The additional carbon dioxide emission of a steam generator in the inventive example relative to comparative example 2 is thus markedly overcompensated.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

### LIST OF REFERENCE NUMERALS

1, 2 Process
10 Natural gas source
11 Compressor
12 HDS unit
13 ATR reactor
14 Deoxygenation unit
15 Fired heating means
16 Water source
17 Water treatment unit
18 Electrolyzer
19 Methanol synthesis unit
20 Distillation unit
21 Methanol product
22 PSA unit
23 SMR unit
24 SMR firing
25 Thermal recovery unit
30 to 54 Conduit
60, 61 Heat flux

1. A process for producing synthesis gas comprising the steps of:
(a) providing a sulfur-containing hydrocarbon stream;
(b) providing an electrolytically produced hydrogen stream;
(c) supplying a portion of the electrolytically produced hydrogen stream to at least a portion of the sulfur-containing hydrocarbon stream to obtain a hydrogen-enriched sulfur-containing hydrocarbon stream;
(d) desulfurizing the stream obtained according to step (c) in a hydrodesulfurization unit to obtain a sulfur-free hydrocarbon stream;
(e) supplying a portion of the electrolytically produced hydrogen stream to at least a portion of the stream obtained according to step (d) to obtain a hydrogen-enriched sulfur-free hydrocarbon stream;
(f) converting at least a portion of the stream obtained according to step (e) into a synthesis gas stream in the presence of oxygen as oxidant in a reforming step.

2. The process according to claim 1, wherein the electrolytically produced hydrogen stream contains oxygen as a concomitant, wherein
no oxygen is removed from the portion of the electrolytically produced hydrogen stream supplied to the sulfur-free hydrocarbon stream according to step (e) and
the oxygen is removed from the portion of the electrolytically produced hydrogen stream supplied to the sulfur-containing hydrocarbon stream according to step (c).

3. The process according to claim 1, wherein
the reforming step comprises an autothermal reforming or a partial oxidation of the stream obtained according to step (e).

4. The process according to claim 1, wherein
a portion of the stream obtained according to step (d) is converted into a synthesis gas substream in an endothermic steam reforming step and
a portion of the stream obtained according to step (d) is enriched with hydrogen according to step (e) to obtain a hydrogen-enriched sulfur-free hydrocarbon substream and
the aforementioned substreams are combined and converted into the synthesis gas stream in a reforming step in the presence of oxygen as oxidant according to step (f).

5. The process according to claim 4, wherein a portion of the electrolytically produced hydrogen stream provided according to step (b) is utilized as fuel in the endothermic steam reforming step.

6. The process according to claim 5, wherein the electrolytically produced hydrogen stream contains oxygen as a concomitant and in that no oxygen is removed from the portion of the electrolytically produced hydrogen stream utilized as fuel in the endothermic steam reforming step.

7. The process according to claim 5, wherein the portion of the electrolytically produced hydrogen stream utilized as fuel is combined with a portion of the sulfur-containing hydrocarbon stream provided according to step (a) to obtain a hydrogen- and hydrocarbon-containing mixed fuel stream which is utilized as fuel in the endothermic steam reforming step.

8. The process according to claim 1, wherein an amount of electrolytically produced hydrogen supplied to the sulfur-free hydrocarbon stream according to step (e) is adjusted such that a synthesis gas stream obtained according to step (f) has a stoichiometry number SN of 1.9 to 2.5, wherein
SN=n(H₂)−n(CO₂)/n(CO)+n(CO₂), with n in [mol].

9. The process according to claim 1, wherein the electrolytically produced hydrogen stream contains 0.01% to 5% by volume of oxygen as a concomitant.

10. The process according to claim 1, wherein the process comprises providing an electrolytically produced oxygen stream, wherein the electrolytically produced oxygen stream is used as oxidant in step (f).

11. The process according to claim 1, further comprising providing an oxygen stream produced by air separation, wherein the oxygen stream produced by air separation is used as oxidant in step (f).

12. The process according to claim 1, wherein the conversion of at least a portion of the stream obtained according to step (e) in the presence of oxygen as oxidant in a reforming step to afford a synthesis gas stream according to step (f) is carried out with additional supply of steam.

13. The process according to claim 1, wherein the electrolytically produced hydrogen stream is partially supplied to the synthesis gas stream produced according to step (f).

14. A process for producing methanol comprising the process for producing synthesis gas according to claim 1, further comprising the step of reacting the synthesis gas stream over a solid methanol synthesis catalyst to afford raw methanol, wherein the raw methanol comprises at least methanol (CH₃OH) and water.

15. The process according to claim 14, wherein the raw methanol is separated into pure methanol and water in a thermal separation process, wherein the thermal separation process affords a carbon-containing tail gas stream, wherein the carbon-containing tail gas stream is utilized as fuel in a heating apparatus for preheating the stream obtained according to step (e) before said stream is converted into synthesis gas according to step (f).

16. The process according to claim 14, wherein the water separated in the thermal separation process is used as starting material for the electrolytically produced hydrogen stream.

17. The process according to claim 14, wherein reacting the synthesis gas stream over the solid methanol synthesis catalyst to afford raw methanol generates a residual gas stream containing synthesis gas unconverted into raw methanol, wherein a portion of the residual gas stream is separated as a purge gas stream and wherein the purge gas stream is supplied to a hydrogen recovery apparatus to produce a non-electrolytically produced hydrogen stream and
the non-electrolytically produced hydrogen stream is at least partially additionally supplied to the sulfur-containing hydrocarbon stream to obtain the hydrogen-enriched sulfur-containing hydrocarbon stream and/or
the non-electrolytically produced hydrogen stream is at least partially additionally supplied to the stream obtained according to step (d) to obtain the hydrogen-enriched sulfur-free hydrocarbon stream.
